(19) United States
(12) Patent Application Publication
(10) Pub. No.: US 2022/0104995 A1
(43) Pub. Date: Apr. 7, 2022

(54) MASSAGE ROLLER ASSEMBLY WITH INFRARED LIGHT THERAPY

(71) Applicant: Therabody, Inc., Los Angeles, CA (US)

(72) Inventors: Jaime Sanchez Solana, Los Angeles, CA (US); Eduardo Merino, Los Angeles, CA (US); Jason Wersland, Los Angeles, CA (US); Benjamin Nazarian, Los Angeles, CA (US)

(21) Appl. No.: 17/525,020

(22) Filed: Nov. 12, 2021

Related U.S. Application Data
(63) Continuation-in-part of application No. 17/491,867, filed on Oct. 1, 2021.
(60) Provisional application No. 63/086,312, filed on Oct. 1, 2020.
(60) Provisional application No. 63/112,850, filed on Nov. 12, 2020.

(51) Int. Cl. A61H 15/02; A61H 15/00

(52) U.S. Cl. CPC A61H 15/02; A61H 15/0092; A61H 2201/10; A61H 2015/0042; A61H 2015/0014

(57) ABSTRACT

A massage roller assembly that includes an outer roller surface, and a plurality of infrared light members positioned adjacent to the outer roller surface and configured to direct infrared light outwardly. The massage roller assembly can be cylindrical, spherical or other shape.

### CROSS REFERENCE TO RELATED APPLICATIONS

This application is a continuation-in-part of U.S. patent application Ser. No. 17/491,867, filed Oct. 1, 2021, which claims the benefit of U.S. Provisional Patent Application No. 63/086,312, filed on Oct. 1, 2020. This application also claims the benefit of U.S. Provisional Patent Application No. 63/112,850, filed Nov. 12, 2020. The entireties of all applications listed above are incorporated by reference herein.

### FIELD OF THE INVENTION

The present invention relates generally to a massage roller, and more particularly to a massage roller with infrared lighting.

### BACKGROUND OF THE INVENTION

Massage rollers that are typically made of or include foam are often used before or after exercise as a self-myofascial release technique to help relieve muscle tightness, soreness, and inflammation, and increase joint range of motion. Light in the infrared range of the spectrum can penetrate to reach muscle tissue and nerves. Infrared lights have a longer wavelength than red lights, so they are best suited to reach deep inside the body's muscles. As the muscle's cells absorb energy from infrared lights, they become more active and increase blood flow. Increased blood flow is shown to help reduce inflammation, and this action may also support natural cellular regeneration. This extra support speeds up the body's natural healing processes and has been shown to help promote wound or injury healing. Infrared light can also help speed up the muscle recovery process and help those who struggle with sore and tight muscles or achy bones. Devices that include infrared light therapy are known. For example, see U.S. Patent Publication No. 2016/0129279 and U.S. Pat. No. 8,900,283, the entireties of which are incorporated by reference herein.

The background description disclosed anywhere in this patent application includes information that may be useful in understanding the present invention. It is not an admission that any of the information provided herein is prior art or relevant to the presently claimed invention, or that any publication specifically or implicitly referenced is prior art.

### SUMMARY OF THE PREFERRED EMBODIMENTS

In accordance with a first aspect of the present invention there is provided a massage roller assembly that includes an outer roller surface, and a plurality of infrared light members positioned adjacent to the outer roller surface and configured to direct infrared light outwardly (e.g. radially outwardly). In a preferred embodiment, the massage roller assembly includes a main body portion that includes a plurality of light recesses defined therein. Each light recess includes at least one of the plurality of infrared light members therein. Preferably, the massage roller assembly also includes a plurality of light covers that are positioned to cover the infrared light members such that light can be emitted through the light covers.

In a preferred embodiment, the massage roller assembly includes a main body portion and an outer cover with a plurality of openings defined therethrough. The openings are positioned to allow light emitted by the plurality of infrared light members to be directed therethrough. Preferably, the plurality of openings each include a light cover disposed therein. In a preferred embodiment, the main body portion defines a roller interior and the infrared light members are electrically communicated with a battery positioned in the roller interior. In a preferred embodiment, the plurality of infrared light members are arranged parallel to a roller axis (lengthwise).

In a preferred embodiment, the present invention is a massage roller assembly that includes infrared lights for providing therapy to the user. In a preferred embodiment, the infrared lights are positioned on the outer surface of the cylindrical main body portion so that the light can shine outwardly. The infrared lights can be positioned as desired and anywhere on the outer surface. The infrared lights may be positioned in sets or in various quadrants or sections so that one or more portions of the outer surface include infrared therapy and others do not. In a preferred embodiment, the infrared lights are LEDs. However, the infrared lights can be any known type of light. Preferably, the LEDs are housed in a recess in the cylindrical main body portion and are covered by a transparent cover or lens.

The present invention is a massage or foam roller that includes infrared or far infrared light therapy (the light emitted is in the infrared and/or far infrared light spectrum. It will be appreciated that the massage roller can be a non-vibrating foam roller, a vibrating foam roller, such as that disclosed in U.S. Patent Publication No. 2016/0113841, U.S. Pat. No. 9,968,513, U.S. Patent Publication No. 2021/0128399 or U.S. patent application Ser. No. 17/491,867, filed Oct. 1, 2021 (the “'867 application”), all of which are incorporated by reference herein in their entireties, or another type of foam roller. The roller can also include lights in the red or blue spectrums or a combination of any of the types of therapeutic lights discussed herein. The light

In a preferred embodiment, the massage roller assembly includes buttons and a display screen (can be a touch screen) on an end thereof for stopping, starting, activating, etc. The screen can also include other functions. The device can include other controllers, such as a thumbwheel or rolling button positioned near the touch screen/on off button to allow the user to scroll or navigate through the different functions.

In a preferred embodiment, the massage roller assembly includes a touch screen, a center button, for turning the device on and off and a ring/rocker button that provides the ability to scroll left and right (e.g., to the preset treatments discussed herein) and up and down (e.g., to control the speed, amplitude or frequency). The screen can also be a non-touch screen.

In a preferred embodiment, the massage roller assembly includes a wireless charging assembly or capability that provides the ability to charge the battery without plugging the battery or the device into anything. Preferably, the massage roller assembly can be received in a charging device or stand, e.g., in a vertical orientation. The charging transmitted is located in the stand and the charging receiver is located in the end of the massage roller. In another embodiment, the massage roller assembly can be charged via a wire.

In another embodiment, the roller assembly includes a cylindrical main body portion (or a spherical main body portion), core or shell housing halves, a battery, a motor, a PCB panel, a counterweight (if it is a vibrating roller), a wireless charger assembly (receiver), an end cap, an on/off button and speed buttons that allow the frequency or speed of the vibrations (based on the speed of the motor/counterweight) to be raised or lowered (e.g., five different speeds). The core housing halves define a housing interior where the motor and counterweight are housed. The motor rotates the counterweight, which causes the housing and cylindrical main body portion to vibrate.

In a preferred embodiment, any of the massage roller assemblies discussed herein can be associated with and can be operated by an app or software that runs on a mobile device such as a phone, watch or tablet (or any computer). The app can connect to the massage roller assembly via bluetooth or other connection protocol. For example, for the massage roller assembly can include bluetooth capability in the PCB panel. The app can have any or all of the following functions. Furthermore, any of the functions discussed herein can be added to the touch screen/scroll wheel or button(s) capability directly on the device. If the user walks or is located too far away from the device, the device will not work or activate. The device can be turned on an off using the app as well as the touch screen or button on the device. The app can control the variable speeds (speed or frequency of vibration in vibrating massage roller) or amplitude/stroke (e.g., of any of the massage members). A timer so the device stops after a predetermined period of time. The app can also include different treatment protocols associated therewith. This will allow the user to choose a protocol or area of the body they want to work on. When the start of the protocol is selected, the device will run through a routine. For example, the infrared lights may turn on and off at predetermined times or different strips or sets of light may turn on and off at predetermined times or in a pattern. The device may run at a first RPM for a first period of time and then run at a second RPM for a second period of time. The routines can also include prompts (e.g., haptic feedback) for letting the user to know to move to a new body part. These routines or treatments can be related to recovery, blood flow increase, performance, etc. and can each include a preprogrammed routine. The routines can also prompt or instruct the user to switch massage members or switch to a different quadrant or area of the device that includes different massage members than are currently being used. The prompts can include sounds, haptic feedback (e.g., vibration of the device or mobile device), textual instructions on the app or touch screen, etc. For example, the app may instruct the user to start by rolling on the device in quadrant one. Then the user hits start and the device runs at a first frequency for a predetermined amount of time. The app or device then prompts the user to begin the next step in the routine and instructs the user to change to rolling in quadrant two. The protocols can include control of or prompts for the user to control or change speed/frequency, amplitude, time, on/off, intensity of light, temperature (if the vibrating roller assembly, or outer surface thereof, is heated).

In another embodiment, the massage roller assembly may configured to sense the location of the user when the user is rolling on or placing their weight on the device. For example, the massage roller assembly can include one or more sensors, positioned anywhere on or in the massage roller assembly, that sense the proximity of the user, the weight of the user, pressure or the like so that the location of the user on the massage roller assembly or outer surface thereof can be determined. The outer cover or another layer of the massage roller assembly can include a capacitive or other sensing layer that is configured to sense (via pressure, proximity, weight or the like) where the user is located. The sensors can also be or include one or more gyrsoscopes, accelerometers, etc. The set or sets of light members that the user is located on (or positioned over) can then be powered or turned on. For example, if the user is sensed to be located in a specific section, the light members in that section can turn or be switched on and as the user rolls partially to the next section, both sets can be turned on and as the user rolls completely to the next section, the light members in the first set can be switched off while the lights in the second set continue to emit light. In another example, only the exact light members that the user is overlying or touching (i.e., the outer cover or light cover over those light members) are switched on.

Similar features are disclosed in the '867 application. All features or capabilities of the vibrating motors in the '867 application are applicable to the light members of the present application.

In a preferred embodiment sets of light members are offset circumferentially from one another about the massage roller assemblies. The sets can be circumferentially offset or positioned at any angle (from 1° to 359°) about the circular shape. The sets can be turned on or off around the circle, as desired.

In another embodiment, the massage roller assembly can includes a horizontal plane that remains horizontal when the device rolls during use. During use, the set of light members above the plane are switched on and the set of light members below the plane are off. This can be done to save battery since the user is only rolling on the light members at the top, upper portion or above the plane. Preferably, the plane bifurcates or is located in the center of the massage roller assembly such that a first set or upper set of light members are the upper half and a second or lower set of light members are the lower half. However, it will be appreciated that plane P1 can be moved upwardly or downwardly. For example, the first set or upper set of light members may only be the top or a single strip of light members and the second or lower set of light members may be all others remaining. All these features combine to allow different sets of light members to be turned on and off as desired whether the massage roller assembly is rolling or not.

Any method for determining when to turn the desired light members on and off is within the scope of the present invention. For example, proximity sensors (that sense when the subject portion of the roller is proximate to the user) can be used. Another embodiment can embody the idea of a compass where the needle always points north. In this embodiment, a component that always points or is directed up or down can be utilized. For example, the roller can include a hanging weight, mass or the like that can be disposed inside the inner shell. During rolling, due to gravity, the weight remains in the lower portion of the roller (i.e., it always “points” or hangs downwardly). As a result, it can be determined what portion of the roller is facing upwardly (and can be utilized by a person for rolling thereon) and what portion of the roller is facing downwardly. In turn, as the roller is rotated and used, the light members in the lower portion of the roller are switched off. Magnets or other types of sensors can be used to sense when the weight is in the lower half or keep the device in the lower portion, thus switching off the light members in the lower portion and switching on the light members in the upper portion at the desired time.

The battery may be any type of battery known in the art. For example, the battery may include a rechargeable lithium-ion (LiIon) based battery. In another example, the battery may include a rechargeable nickel metal hydride (NiMH) battery. In yet another example, the battery may include a rechargeable lithium-polymer (LiPo) battery. In some embodiments, the battery includes a nickel-cadmium (NiCad) battery. In one embodiment, the battery uses a non-rechargeable battery.

### BRIEF DESCRIPTION OF THE DRAWINGS

FIG. 1 is a perspective view of a cylindrical massage roller assembly in accordance with a preferred embodiment of the present invention;

FIG. 2 is an exploded perspective view of the massage roller assembly;

FIG. 3 is a cross-sectional end view of the massage roller assembly;

FIG. 4 is a side elevational view of another embodiment of a massage roller assembly;

FIG. 5 is a perspective view of a spherical massage roller assembly in accordance with a preferred embodiment of the present invention; and

FIG. 6 is an exploded perspective view of the massage roller assembly.

Like numerals refer to like parts throughout the several views of the drawings.

### DETAILED DESCRIPTION OF THE PREFERRED EMBODIMENTS

The following description and drawings are illustrative and are not to be construed as limiting. Numerous specific details are described to provide a thorough understanding of the disclosure. However, in certain instances, well-known or conventional details are not described in order to avoid obscuring the description. References to one or an embodiment in the present disclosure can be, but not necessarily are references to the same embodiment; and, such references mean at least one of the embodiments. If a component is not shown in a drawing then this provides support for a negative limitation in the claims stating that that component is “not” present. However, the above statement is not limiting and in another embodiment, the missing component can be included in a claimed embodiment.

Reference in this specification to “one embodiment,” “an embodiment,” “a preferred embodiment” or any other phrase mentioning the word “embodiment” means that a particular feature, structure, or characteristic described in connection with the embodiment is included in at least one embodiment of the-disclosure and also means that any particular feature, structure, or characteristic described in connection with one embodiment can be included in any embodiment or can be omitted or excluded from any embodiment. The appearances of the phrase “in one embodiment” in various places in the specification are not necessarily all referring to the same embodiment, nor are separate or alternative embodiments mutually exclusive of other embodiments. Moreover, various features are described which may be exhibited by some embodiments and not by others and may be omitted from any embodiment. Furthermore, any particular feature, structure, or characteristic described herein may be optional. Similarly, various requirements are described which may be requirements for some embodiments but not other embodiments. Where appropriate any of the features discussed herein in relation to one aspect or embodiment of the invention may be applied to another aspect or embodiment of the invention. Similarly, where appropriate any of the features discussed herein in relation to one aspect or embodiment of the invention may be optional with respect to and/or omitted from that aspect or embodiment of the invention or any other aspect or embodiment of the invention discussed or disclosed herein.

The terms used in this specification generally have their ordinary meanings in the art, within the context of the disclosure, and in the specific context where each term is used. Certain terms that are used to describe the disclosure are discussed below, or elsewhere in the specification, to provide additional guidance to the practitioner regarding the description of the disclosure. For convenience, certain terms may be highlighted, for example using italics and/or quotation marks: The use of highlighting has no influence on the scope and meaning of a term; the scope and meaning of a term is the same, in the same context, whether or not it is highlighted.

It will be appreciated that the same thing can be said in more than one way. Consequently, alternative language and synonyms may be used for any one or more of the terms discussed herein. No special significance is to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and is not intended to further limit the scope and meaning of the disclosure or of any exemplified term. Likewise, the disclosure is not limited to various embodiments given in this specification.

Without intent to further limit the scope of the disclosure, examples of instruments, apparatus, methods and their related results according to the embodiments of the present disclosure are given below. Note that titles or subtitles may be used in the examples for convenience of a reader, which in no way should limit the scope of the disclosure. Unless otherwise defined, all technical and scientific terms used herein have the same meaning as commonly understood by one of ordinary skill in the art to which this disclosure pertains. In the case of conflict, the present document, including definitions, will control.

It will be appreciated that terms such as “front,” “back,” “top,” “bottom,” “side,” “short,” “long,” “up,” “down,” “aft,” “forward,” “inboard,” “outboard” and “below” used herein are merely for ease of description and refer to the orientation of the components as shown in the figures. It should be understood that any orientation of the components described herein is within the scope of the present invention.

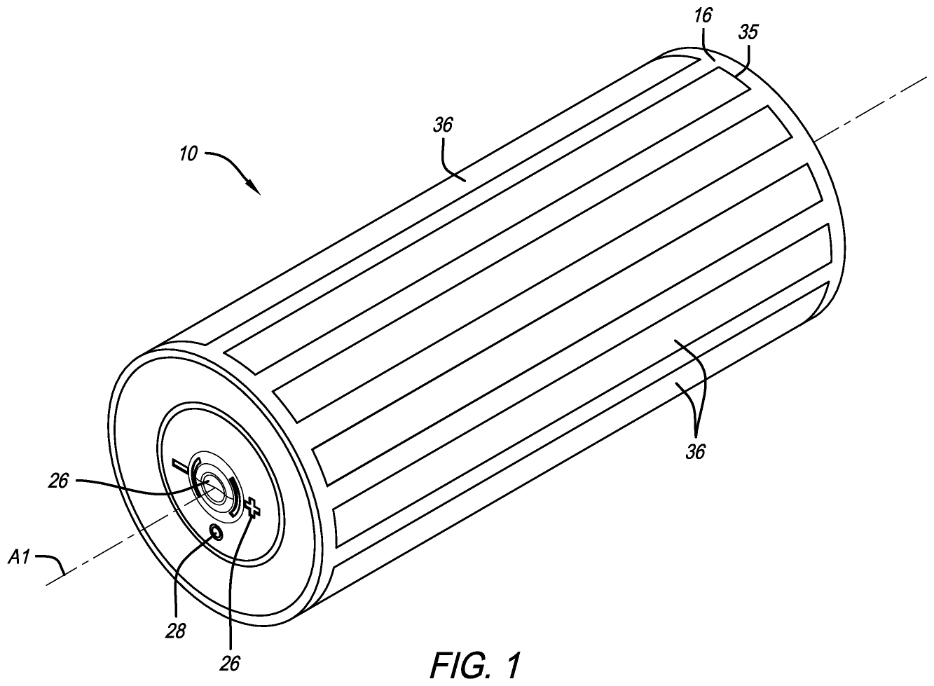

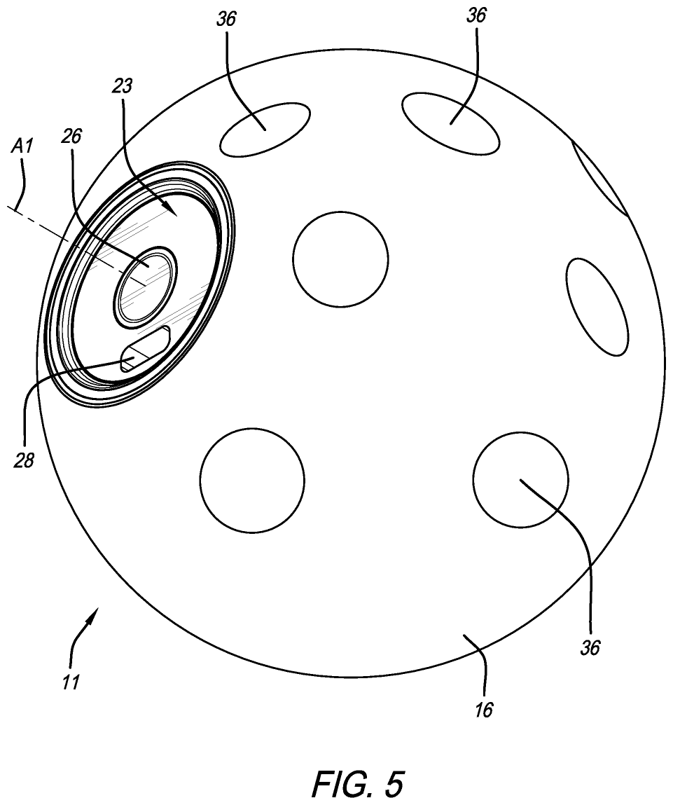

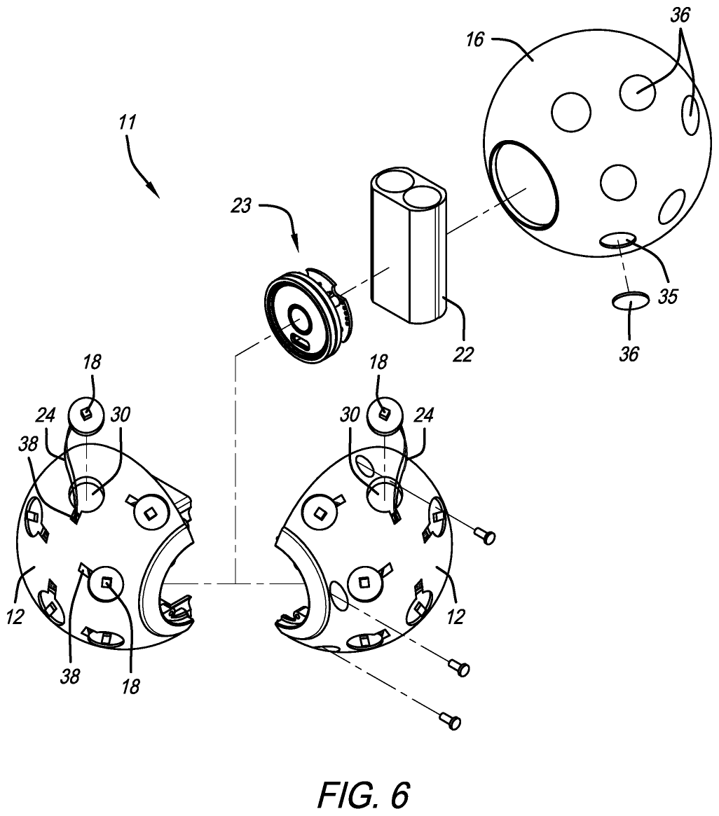

Referring now to the drawings, which are for purposes of illustrating the present invention and not for purposes of limiting the same, the drawings show a massage roller assembly that includes therapeutic lighting. FIGS. 1-6 show embodiments of two massage roller assemblies that include infrared lighting distributed around the massage roller assembly and that provide infrared light therapy while using the roller assembly. FIGS. 1-4 show a cylindrical massage roller assembly 10 and FIGS. 5-6 show a spherical massage roller assembly 11. It will be appreciated that other shapes are within the scope of the present invention. For example, see U.S. patent application Ser. No. 29/745,555, the entirety of which is incorporated by reference herein, and which shows a peanut shaped massage roller assembly.

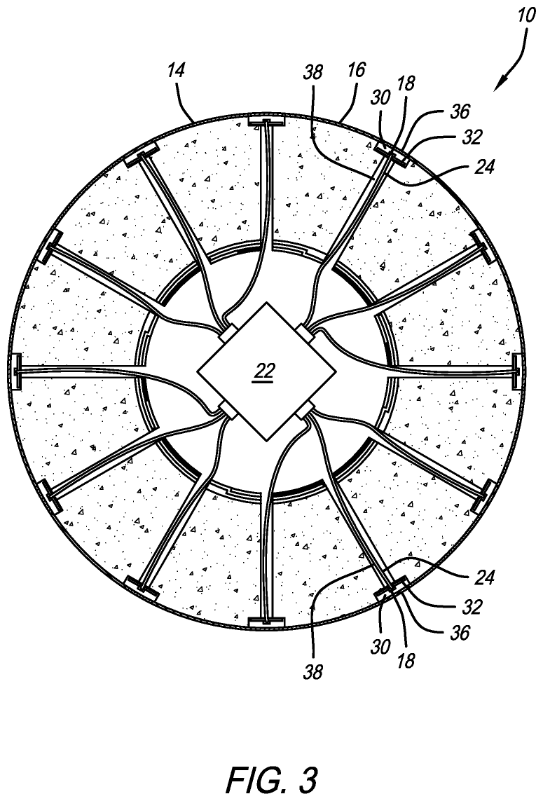

As shown in FIGS. 1-3, the massage roller assembly 10 generally includes a main body portion or inner shell 12 that defines a roller axis A1 and includes an outer roller surface 14, an outer cover 16, and a plurality of infrared or therapeutic light members 18. The outer roller surface 14 of the inner shell 12 is not necessarily the surface used for contact with a user. As shown in FIG. 3, the outer roller surface 14 is inside the outer cover 16. However, in another embodiment the outer cover can be omitted. In another embodiment, the outer roller surface is the outer surface of the outer cover 16. In a preferred embodiment, the inner shell 12 that may include separate halves or portions. The inner shell 12 preferably houses the battery 22 and the motor and associated components if the roller is a vibrating roller.

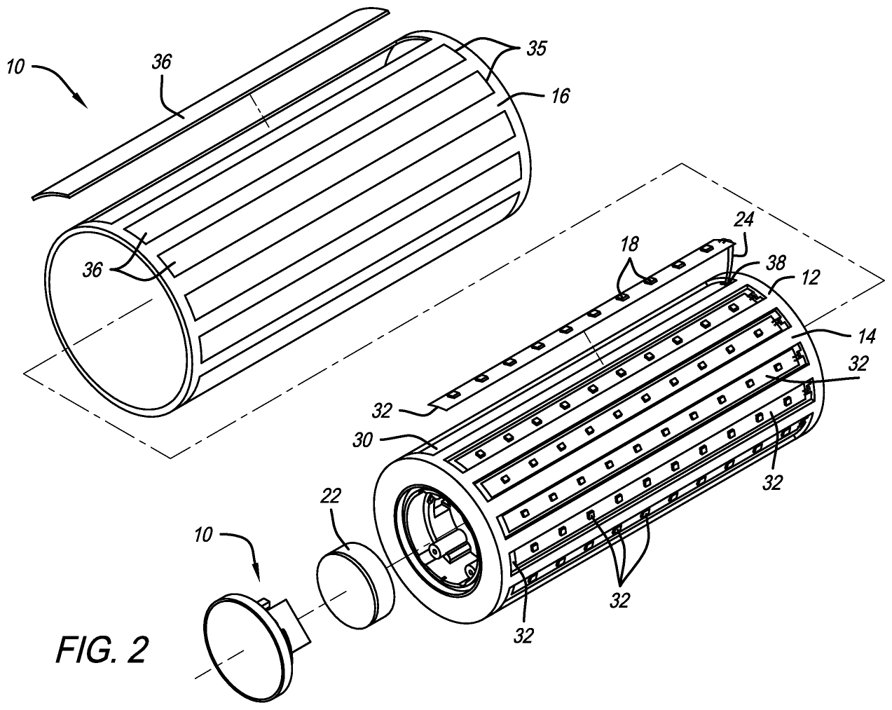

As shown in FIGS. 5-6, the massage roller assembly 11 generally includes a spherical main body portion or shell 12 that defines a roller axis A1 and an outer cover 16 having an outer roller surface 14. All description related to the cylindrical roller assembly 10 is applicable to the spherical massage roller assembly 11 and vice versa. The shell 12 may be a single hollow piece (see FIG. 2) or may include separate halves or portions (see FIG. 6). The shell 12 preferably houses one or more batteries 22 for powering the device and the electronics of the device, which may include a controller, PCB, memory or other components for controlling and operating the device. The housing halves can be held together with threaded fasteners, as shown in FIG. 6.

For example, the massage roller assembly 10 may include an electronics assembly 23 located at the end of the cylinder (see FIGS. 1 and 2) or within the shell 12 and/or anywhere else in the massage roller assembly. See, e.g., exemplary control buttons 26. A display or screen (touch screen or non-touch screen) can also be included. The buttons can be used for on/off, cycling through different lighting patterns or intensities, changing speeds of vibration, connecting to Bluetooth, etc.). A charging and/or connecting port 28 can also be included.

As shown in FIG. 2, in a preferred embodiment, the shell 12 includes light recesses 30 that are configured to receive the infrared light members 18. In FIGS. 2 and 3, the light members 18 are shown in or on strips 32 that are electrically connected via wires 24 to the battery 22. The light recesses 30 are preferably open to the outside of the shell 12, as shown in FIGS. 2 and 6. Separate light recess 30 can be used for each light, if desired. In a preferred embodiment, the outer cover 16 includes openings 35 defined therethrough that include a lens or light cover 36 positioned therein. As shown in FIG. 3, the light covers 36 are positioned over the infrared light members 18 so that light is emitted through the light covers 36 during use. The covers can be transparent or opaque, as desired. In another embodiment, the light recesses can be omitted. In another embodiment, the outer cover can be omitted and the covers can be positioned in the light recesses 30 in the main body portion 12 to cover the light members 18. The shell 12 preferably includes wire openings 38 defined therein for directing wires into the interior of the shell 12. FIGS. 2 and 6 show one or more of the light members 18 exploded from the shell 12 and shows the wires 24 exploded therewith, but extending into the wire openings 38. As shown in FIG. 3, if the inner shell or main body portion is thick, the wire openings 38 may be tunnels that extend into the interior to guide the wires to the appropriate location so that the lights 18 are electrically and/or data communicated with the PCB, battery 22, electronics assembly 23, etc., as appropriate. The outer cover 16, together with the light covers 36, surround and cover the shell 12 and light members 18 for a user to roll on. The lights 18 transmit or emit light through the light covers 36 and radially outwardly from the massage roller assembly.

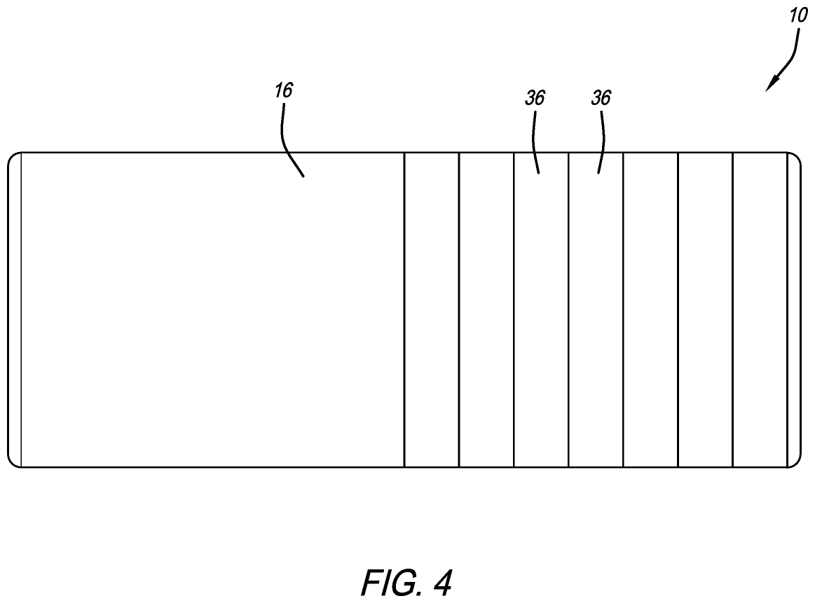

It will be appreciated that the light members 18 can be arranged in any desirable pattern so that they emit lights outwardly from the massage roller. For example, as shown in FIG. 4, the light covers 36 (and the associated lights) may only be included on a portion of the massage roller. Furthermore, FIG. 4 shows the elongated light covers 36 extending circumferentially as opposed to lengthwise or axially as is shown in FIG. 1. FIG. 5 shows lights and the associated light covers 36 distributed individually around the spherical massage roller assembly 11. The cylindrical massage roller assembly 10 can also include this feature. The outer circumference or outer circumferential surface of the massage roller assembly 10 or 11 or outer cover 16 thereof may include any number of lights or light sections (e.g., 1-1000 or any number in between). Furthermore, the lights can be grouped into any number of sets (a set can include from 1 to all lights) that can be turned on independently or as desired. The electronics assembly can include the ability to cycle through different light patterns or intensities where certain one or more sets emit light for a first period of time and then one or more different sets emit light for a second period of time, etc.

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof, means any connection or coupling, either direct or indirect, between two or more elements; the coupling of connection between the elements can be physical, logical, or a combination thereof. Additionally, the words “herein,” “above,” “below,” and words of similar import, when used in this application, shall refer to this application as a whole and not to any particular portions of this application. Where the context permits, words in the above Detailed Description of the Preferred Embodiments using the singular or plural number may also include the plural or singular number respectively. The word “or” in reference to a list of two or more items, covers all of the following interpretations of the word: any of the items in the list, all of the items in the list, and any combination of the items in the list.

The above-detailed description of embodiments of the disclosure is not intended to be exhaustive or to limit the teachings to the precise form disclosed above. While specific embodiments of and examples for the disclosure are described above for illustrative purposes, various equivalent modifications are possible within the scope of the disclosure, as those skilled in the relevant art will recognize. Further, any specific numbers noted herein are only examples: alternative implementations may employ differing values, measurements or ranges.

Although the operations of any method(s) disclosed or described herein either explicitly or implicitly are shown and described in a particular order, the order of the operations of each method may be altered so that certain operations may be performed in an inverse order or so that certain operations may be performed, at least in part, concurrently with other operations. In another embodiment, instructions or sub-operations of distinct operations may be implemented in an intermittent and/or alternating manner.

The teachings of the disclosure provided herein can be applied to other systems, not necessarily the system described above. The elements and acts of the various embodiments described above can be combined to provide further embodiments. Any measurements or dimensions described or used herein are merely exemplary and not a limitation on the present invention. Other measurements or dimensions are within the scope of the invention.

Any patents and applications and other references noted above, including any that may be listed in accompanying filing papers, are incorporated herein by reference in their entirety. Aspects of the disclosure can be modified, if necessary, to employ the systems, functions, and concepts of the various references described above to provide yet further embodiments of the disclosure.

These and other changes can be made to the disclosure in light of the above Detailed Description of the Preferred Embodiments. While the above description describes certain embodiments of the disclosure, and describes the best mode contemplated, no matter how detailed the above appears in text, the teachings can be practiced in many ways. Details of the system may vary considerably in its implementation details, while still being encompassed by the subject matter disclosed herein. As noted above, particular terminology used when describing certain features or aspects of the disclosure should not be taken to imply that the terminology is being redefined herein to be restricted to any specific characteristics, features or aspects of the disclosure with which that terminology is associated. In general, the terms used in the following claims should not be construed to limit the disclosures to the specific embodiments disclosed in the specification unless the above Detailed Description of the Preferred Embodiments section explicitly defines such terms. Accordingly, the actual scope of the disclosure encompasses not only the disclosed embodiments, but also all equivalent ways of practicing or implementing the disclosure under the claims.

While certain aspects of the disclosure are presented below in certain claim forms, the inventors contemplate the various aspects of the disclosure in any number of claim forms. For example, while only one aspect of the disclosure is recited as a means-plus-function claim under 35 U.S.C. § 112,¶6, other aspects may likewise be embodied as a means-plus-function claim, or in other forms, such as being embodied in a computer-readable medium. (Any claims intended to be treated under 35 U.S.C. § 112, ¶6 will include the words “means for”). Accordingly, the applicant reserves the right to add additional claims after filing the application to pursue such additional claim forms for other aspects of the disclosure.

Accordingly, although exemplary embodiments of the invention have been shown and described, it is to be understood that all the terms used herein are descriptive rather than limiting, and that many changes, modifications, and substitutions may be made by one having ordinary skill in the art without departing from the spirit and scope of the invention.

What is claimed is:

1. A massage roller assembly comprising:
an outer roller surface, and
a plurality of infrared light members positioned adjacent to the outer roller surface and configured to direct infrared light outwardly.

2. The massage roller assembly of claim 1 further comprising a main body portion that includes a plurality of light recesses defined therein, wherein each light recess includes at least one of the plurality of infrared light members therein.

3. The massage roller assembly of claim 2 further comprising a plurality of light covers that are positioned to cover the infrared light members such that light can be emitted through the light covers.

4. The massage roller assembly of claim 1 further comprising a main body portion and an outer cover with a plurality of openings defined therethrough, wherein the openings are positioned to allow light emitted by the plurality of infrared light members to be directed therethrough.

5. The massage roller of claim 4 wherein the plurality of openings each include a light cover disposed therein.

6. The massage roller of claim 5 wherein the main body portion includes a plurality of light recesses defined therein, wherein each light recess includes at least one of the plurality of infrared light members therein.

7. The massage roller assembly of claim 6 wherein the main body portion defines a roller interior, and wherein the infrared light members are electrically communicated with a battery positioned in the roller interior.

8. The massage roller assembly of claim 1 comprising a cylindrical shape that defines a roller axis.

9. The massage roller assembly of claim 8 wherein the plurality of infrared light members are arranged parallel to the roller axis.

10. The massage roller assembly of claim 1 comprising a spherical shape.

11. A massage roller assembly comprising:
a main body portion that includes a plurality of light recess defined therein, wherein each light recess includes at least one infrared light member disposed therein, wherein the main body portion defines a roller interior including a battery positioned therein, and
an outer cover with a plurality of openings defined therethrough, wherein each opening includes a light cover disposed therein, wherein the infrared light members are positioned adjacent to the light covers and are configured to direct infrared light outwardly, wherein the infrared light members are electrically communicated with the battery.
